(19) United States
(12) Patent Application Publication
(10) Pub. No.: US 2025/0172719 A1
(43) Pub. Date: May 29, 2025

(54) SYSTEM AND METHOD FOR GENERATING REGIONAL PETROPHYSICAL MODELS

(71) Applicant: Chevron U.S.A. Inc., San Ramon, CA (US)

(72) Inventors: Haijing WANG, Sugar Land, TX (US); Luisa Carolina CROUSSE, Houston, TX (US); Sergio Alejandro CUERVO, Buenos Aires (AR); Ronald L. BROWN, Lekki, Lagos (NG); Timothy B. FISCHER, Fort Collins, CO (US); Mason Cole EDWARDS, Houston, TX (US); Andrew B. TIPTON, Houston, TX (US); Michael Christopher CHESHIRE, Richmond, TX (US); Scott Alan HANSON, Houston, TX (US); Ali WUSHUR, Houston, TX (US)

(73) Assignee: Chevron U.S.A. Inc., San Ramon, CA (US)

(21) Appl. No.: 18/947,823

(22) Filed: Nov. 14, 2024

Related U.S. Application Data
(60) Provisional application No. 63/603,865, filed on Nov. 29, 2023.

(51) Int. Cl. G01V 20/00; G16C 60/00

(52) U.S. Cl. CPC G01V 20/00; G16C 60/00

(57) ABSTRACT

A method is described for generating a regional petrophysical model. The method receives high-definition (HD) well logs; builds at least one HD petrophysical model based on the HD well logs; and builds a regional petrophysical model based on the at least one HD petrophysical model. It may then apply the regional petrophysical model to wells with basic logging suites. The method is executed by a computer system.

### CROSS-REFERENCE TO RELATED APPLICATIONS

This application claims the benefit of U.S. Provisional Application 63/603,865 titled “System and Method for Generating Regional Petrophysical Models” filed Nov. 29, 2023.

### TECHNICAL FIELD

The disclosed embodiments relate generally to techniques for petrophysical interpretation. In particular, the disclosed embodiments relate to techniques for processing various petrophysical data to extrapolate to regional petrophysical models.

### BACKGROUND

Petrophysical interpretation, consisting of rock and fluid characterization using well logs and core analysis, plays an important role throughout field lifecycle from exploration to abandonment.

There are two mainstreams of interpretation methods: deterministic petrophysics and multimineral analysis. Deterministic petrophysics, based primarily on basic well logs including gamma ray, neutron porosity, bulk density, photoelectric factor, and acoustic logs, assumes simple lithology and utilizes empirical linear and non-linear equations to solve for basic petrophysical properties, such as lithology, porosity, saturation, and permeability, from input well logs. It is a sequential process with the results of each step determined by the results from prior steps. The multi-dimensional interaction between measurements and answers is not reflected. While deterministic petrophysics has been applied to hydrocarbon reservoirs for decades, it is challenging to use it in formations with complex lithologies. Multimineral analysis is an alternative interpretation method for complex lithology. It consists of a petrophysical model that includes mineral and fluid components, as well as their tool response parameters. With fixed tool response parameters, multimineral analysis uses probabilistic method to reconstruct the measured tool response by optimizing the volumes.

The advantages of multimineral analysis go beyond solving for improving the petrophysical solution in areas of complex lithologies. Multimineral analysis often requires more geological knowledge, which leads to more robust interpretation as well as better integration and communication with other disciplines. It can also incorporate a multitude of log data in an integrated way, such as nuclear spectroscopy, nuclear magnetic resonance (NMR), and dielectric logs. The direct incorporation of advanced logs in multimineral analysis can greatly enhance our capability to solve for complex mineralogy, organic matter, porosity, and saturation.

Whenever available, the interpretation results are calibrated against or integrated with core analysis, which are often taken as a ground truth. Core analysis is based on physical measurement used to be compared with petrophysical interpretation answers (such as lithology, porosity, and saturation), not always possible with basic well logs such as bulk density and neutron porosity. It remains difficult to verify whether input well logs are of good quality, or the log response for each rock and fluid component used in the interpretation is appropriate.

Several forms of core sample can be acquired from the subsurface: whole cores, rotary side-wall cores (RSWC), and cuttings. Whole cores taken in exploration wells typically measure no more than a few hundred feet. Desired core measurements are then performed on core plugs cut from whole core at selected depth points. RSWCs are taken from downhole at selected and discrete depths. The sidewall cores are not taken from continuous depths but can potentially cover wider depth intervals. Cuttings are collected on the surface at varying frequencies, although it is often challenging to correlate cuttings depth to logs or core. In general, the core analysis provides non-continuous calibration to well logs within certain depth interval.

A typical regional petrophysical model is based on basic well log suites, such as gamma ray, neutron porosity, bulk density, and resistivity, to maximize its applicability in as many wells as possible. The basic logs are fed into multimineral petrophysical models in which the log responses of each rock and fluid constituents are provided. Such log responses are often calibrated against various lab measurements based on available core and cuttings. By varying the volume of each rock and fluid constituents, the difference between predicted and measured log responses is minimized under certain constraints to determine the most likely volumetric composition of the subsurface formation. This method works fine in conventional reservoirs where the rock matrix consists of relatively simple lithologies such as sand and shale. However, it is still a challenging task to build a robust regional petrophysical model in unconventional resources due to complex mineralogy, organic matter, and low porosity. For instance, current regional petrophysical model is solving for a simplified group of minerals consisting of illite, quartz, calcite, pyrite, and kerogen, constrained by the limited number of inputs from basic logging suites. However, such a model often produces questionable results in some areas and formations, such as overpredicting calcite and pyrite, and underpredicting organic matter, which leads to more uncertainties in porosity and saturation. FIG. 1 demonstrates that current regional models overestimate calcite and underestimate organic matter in some regions and formations. For instance, on the left track of FIG. 1, the regional multimineral model (as shown in light shade labelled as MM_Reg), shows almost 100 weight percents of calcite near the bottom of Unit 2. However, the quantitative X-ray diffraction (QXRD) based on core samples, shown as solid dots, indicated maximum of 80 weight percents of calcite in the same intervals. On the right track of FIG. 1, the regional multimineral model (as shown in dark shade labelled as MM_Reg) underestimated kerogen as compared with the weight fraction of organic matters obtained from Fourier transform infrared (FTIR) spectroscopy analysis of cuttings samples (as shown in solid dots) in lower Unit A and upper Unit B. These could have significant impact on rock mechanical properties such as frac (hydraulic fracturing) barrier. More robust and standardized regional models are needed.

There exists a need for methods to build robust regional petrophysical models.

### SUMMARY

In accordance with some embodiments, a method of building a regional petrophysical model is disclosed. In an embodiment, the method receives high-definition (HD) well logs; builds at least one HD petrophysical model based on the HD well logs; and builds a regional petrophysical model based on the at least one HD petrophysical model. In another embodiment, the method may then apply the regional petrophysical model to wells with basic logging suites, or standard-definition (SD) wells.

In another aspect of the present invention, to address the aforementioned problems, some embodiments provide a non-transitory computer readable storage medium storing one or more programs. The one or more programs comprise instructions, which when executed by a computer system with one or more processors and memory, cause the computer system to perform any of the methods provided herein.

In yet another aspect of the present invention, to address the aforementioned problems, some embodiments provide a computer system. The computer system includes one or more processors, memory, and one or more programs. The one or more programs are stored in memory and configured to be executed by the one or more processors. The one or more programs include an operating system and instructions that when executed by the one or more processors cause the computer system to perform any of the methods provided herein.

### BRIEF DESCRIPTION OF THE DRAWINGS

FIG. 1 demonstrates two examples showing that conventional regional models overestimate calcite and underestimate organic matter in some regions and formations;

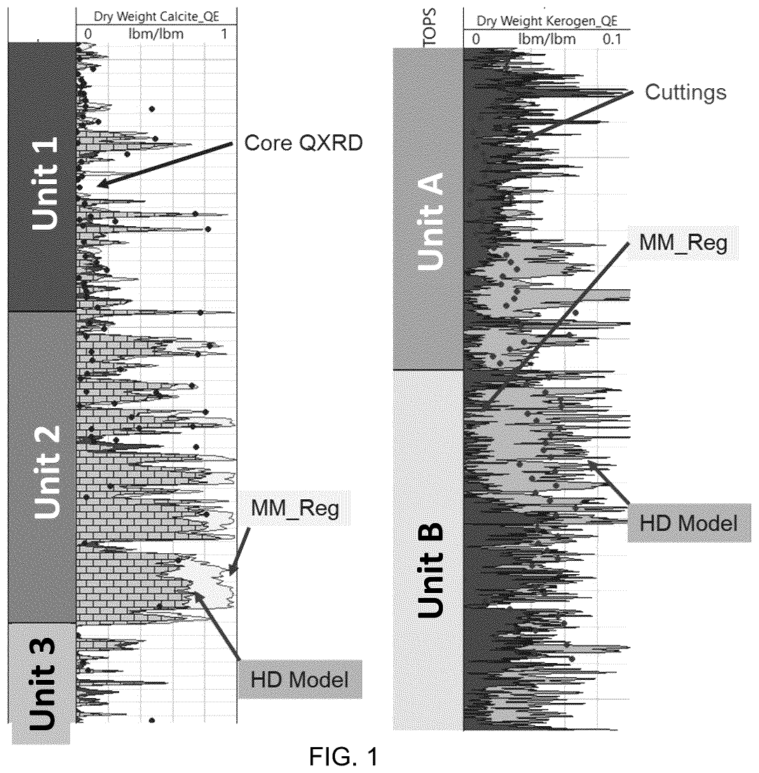

FIG. 2 illustrates the comparison between standard-definition petrophysical interpretation, which uses basic well logs as inputs and solve for a simplified set of minerals, and high-definition petrophysical interpretation by combining both basic and advanced well logs (such as nuclear spectroscopy, NMR, and dielectric logs) and core/cuttings analysis;

FIG. 3 illustrates the process of applying parameters, calibrations, and models from high-definition wells to wells with basic logging suites (standard-definition wells);

FIG. 4 illustrates an example system for developing develop robust regional petrophysical models;

FIG. 5 illustrates an example method for developing develop robust regional petrophysical models;

FIG. 6 is a comparison of elemental dry weights from nuclear spectroscopy logs (continuous curves) with those measured by core chemistry (solid dots);

FIG. 7 is a comparison of calibration coverage among whole core, rotary side-wall cores, and well logs. The left panel shows the weight fraction of calcite in comparison with the same quantity measured in core plugs taken from whole core and from rotatory side-wall cores (RSWC). The right panel illustrate the depth coverage of whole core, RSWC, and well logs;

FIG. 8 is an example of high-definition well interpretation results integrating nuclear spectroscopy, NMR, and dielectric logs, and calibration with core mineralogy, porosity, and saturation measurements;

FIG. 9 is an example of porosity comparison between high-definition and current regional models shown as frequency plots of total porosity;

FIG. 10 is derivation of the relationship between water-phase tortuosity parameter “MN” from dielectric dispersion logging and Archie's parameter cementation exponent (m) and saturation exponent (n);

FIG. 11 is numerical calculation of MN as a function of water saturation with typical porosity values (5, 10, 15 porosity units) under specific assumption of m=2.35, n=2.0; and

FIG. 12 is numerical calculation of MN as a function of water saturation with typical porosity values (5, 10, 15 porosity units) under specific assumption of m=2.1, n=2.0.

Like reference numerals refer to corresponding parts throughout the drawings.

### DETAILED DESCRIPTION OF EMBODIMENTS

Described below are methods, systems, and computer readable storage media that provide a manner of petrophysical interpretation. These embodiments are designed to be of particular use for processing various petrophysical data to extrapolate to regional models. The methods and workflows disclosed herein consist of upscaling well log calibration from point core data using continuous nuclear spectroscopy logs and calibrating multimineral models from high-definition wells to regional models.

Reference will now be made in detail to various embodiments, examples of which are illustrated in the accompanying drawings. In the following detailed description, numerous specific details are set forth in order to provide a thorough understanding of the present disclosure and the embodiments described herein. However, embodiments described herein may be practiced without these specific details. In other instances, well-known methods, procedures, components, and mechanical apparatus have not been described in detail so as not to unnecessarily obscure aspects of the embodiments.

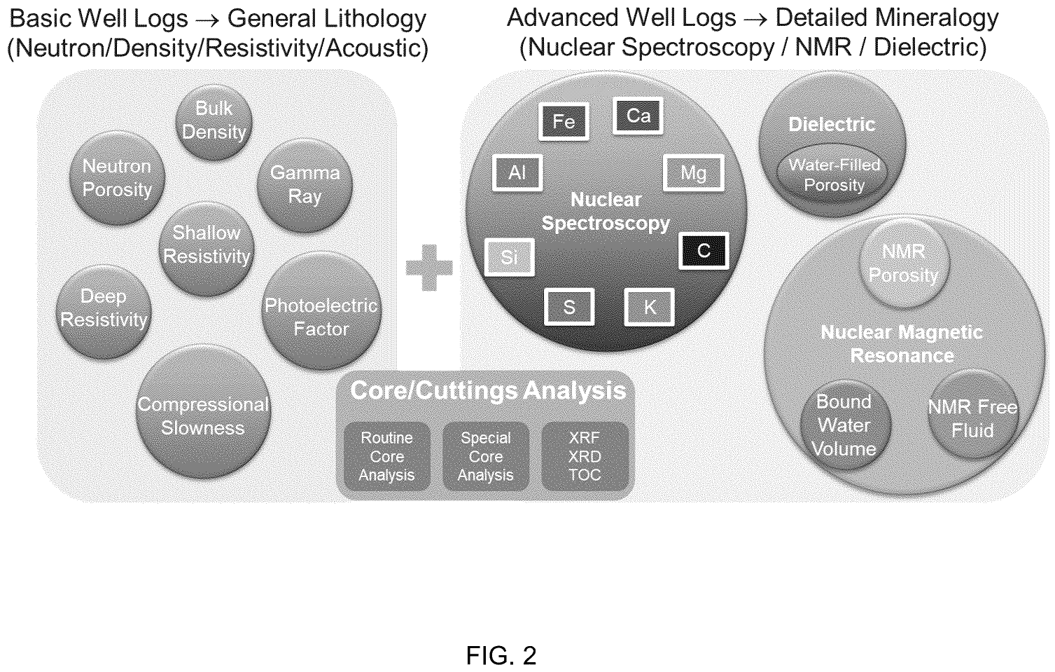

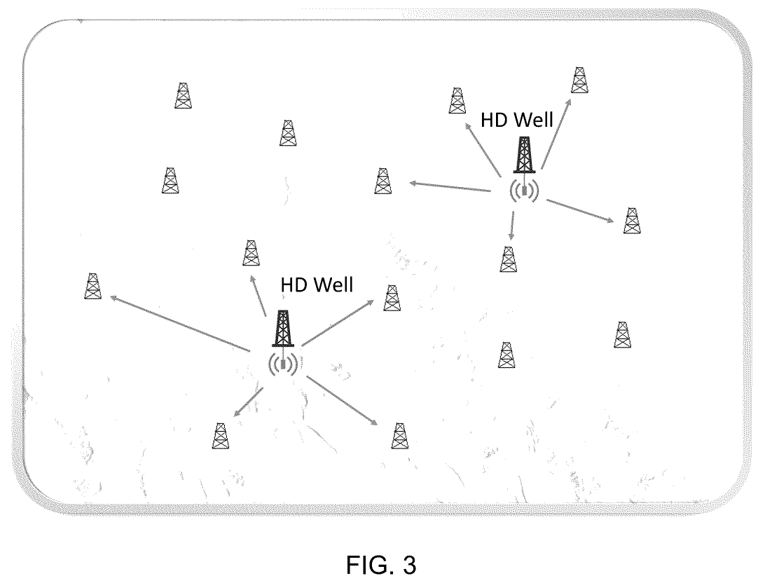

Petrophysical interpretation is essential to reservoir characterization, landing point optimization, and rock mechanical assessment in unconventional reservoirs (unconventional reservoirs include tight rock, shale, carbonate, and the like). A typical regional petrophysical model is based on basic logging suites (a basic logging suite includes one or more of gamma ray, neutron porosity, bulk density, and resistivity) to maximize its applicability in as many wells as possible. However, it is a challenging task to build a robust petrophysical interpretation due to complex mineralogy, organic matter, and low porosity. In recent years, there have been more advanced well logs acquired such as nuclear spectroscopy, nuclear magnetic resonance, and dielectric, in selected wells that can be designated as high-definition (HD) wells (as illustrated by FIG. 2). The present invention fully integrates advanced well logs into a unified interpretation, and anchors on the HD wells to develop a more robust regional petrophysical model across major unconventional assets (as illustrated by FIG. 3).

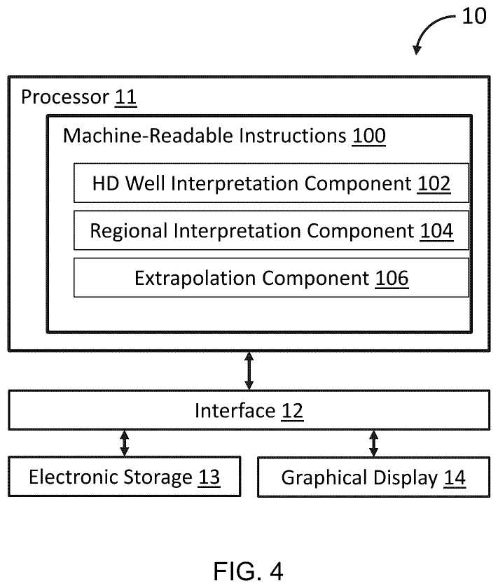

The methods and systems of the present disclosure may be implemented by a system and/or in a system, such as a system 10 shown in FIG. 4. The system 10 may include one or more of a processor 11, an interface 12 (e.g., bus, wireless interface), an electronic storage 13, a graphical display 14, and/or other components. The processor 11 is capable of receiving basic and advanced well logs in high-definition (HD) wells, and generating regional petrophysical models that are using basic well logs as inputs and calibrated against the HD models established in HD wells.

The electronic storage 13 may be configured to include electronic storage medium that electronically stores information. The electronic storage 13 may store software algorithms, information determined by the processor 11, information received remotely, and/or other information that enables the system 10 to function properly. For example, the electronic storage 13 may store information relating to input well logs, and/or other information. For example, the electronic storage 13 may store information relating to output petrophysical models, and/or other information. The electronic storage media of the electronic storage 13 may be provided integrally (i.e., substantially non-removable) with one or more components of the system 10 and/or as removable storage that is connectable to one or more components of the system 10 via, for example, a port (e.g., a USB port, a Firewire port, etc.) or a drive (e.g., a disk drive, etc.). The electronic storage 13 may include one or more of optically readable storage media (e.g., optical disks, etc.), magnetically readable storage media (e.g., magnetic tape, magnetic hard drive, floppy drive, etc.), electrical charge-based storage media (e.g., EPROM, EEPROM, RAM, etc.), solid-state storage media (e.g., flash drive, etc.), and/or other electronically readable storage media. The electronic storage 13 may include one or more non-transitory computer readable storage medium storing one or more programs. The electronic storage 13 may be a separate component within the system 10, or the electronic storage 13 may be provided integrally with one or more other components of the system 10 (e.g., the processor 11). Although the electronic storage 13 is shown in FIG. 4 as a single entity, this is for illustrative purposes only. In some implementations, the electronic storage 13 may comprise a plurality of storage units. These storage units may be physically located within the same device, or the electronic storage 13 may represent storage functionality of a plurality of devices operating in coordination.

The graphical display 14 may refer to an electronic device that provides visual presentation of information. The graphical display 14 may include a color display and/or a non-color display. The graphical display 14 may be configured to visually present information. The graphical display 14 may present information using/within one or more graphical user interfaces. For example, the graphical display 14 may present information relating to high-definition well log interpretations, petrophysical models, and/or other information.

The processor 11 may be configured to provide information processing capabilities in the system 10. As such, the processor 11 may comprise one or more of a digital processor, an analog processor, a digital circuit designed to process information, a central processing unit, a graphics processing unit, a microcontroller, an analog circuit designed to process information, a state machine, and/or other mechanisms for electronically processing information. The processor 11 may be configured to execute one or more machine-readable instructions 100 to facilitate generating regional petrophysical models. The machine-readable instructions 100 may include one or more computer program components. The machine-readable instructions 100 may include a well interpretation component 102, a regional interpretation component 104, an extrapolation component 106, and/or other computer program components.

It should be appreciated that although computer program components are illustrated in FIG. 4 as being co-located within a single processing unit, one or more of computer program components may be located remotely from the other computer program components. While computer program components are described as performing or being configured to perform operations, computer program components may comprise instructions which may program processor 11 and/or system 10 to perform the operation.

While computer program components are described herein as being implemented via processor 11 through machine-readable instructions 100, this is merely for ease of reference and is not meant to be limiting. In some implementations, one or more functions of computer program components described herein may be implemented via hardware (e.g., dedicated chip, field-programmable gate array) rather than software. One or more functions of computer program components described herein may be software-implemented, hardware-implemented, or software and hardware-implemented.

Referring again to machine-readable instructions 100, the high-definition (HD) well interpretation component 102 may be configured to receive HD well logs and build a robust HD petrophysical model and interpretation within the HD well(s).

The regional interpretation component 104 may be configured to use the HD petrophysical model and interpretations from the HD wells to calibrate within the HD wells and for use with basic well log suites. It will establish mineral correlations, constraints, and trends based on HD model and apply to regional models, iteratively verifying the regional models and determining applicable boundaries.

The extrapolation component 106 may be configured to apply regional petrophysical model to wells with basic logging suites, or standard-definition (SD) wells. This will result in a robust regional petrophysical model.

The description of the functionality provided by the different computer program components described herein is for illustrative purposes, and is not intended to be limiting, as any of computer program components may provide more or less functionality than is described. For example, one or more of computer program components may be eliminated, and some or all of its functionality may be provided by other computer program components. As another example, processor 11 may be configured to execute one or more additional computer program components that may perform some or all of the functionality attributed to one or more of computer program components described herein.

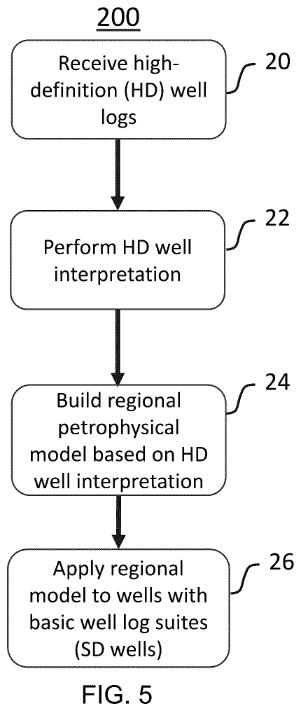

FIG. 5 illustrates an example process 200 for generating regional petrophysical models. At step 20, high-definition (HD) well logs are received. These include, by way of example and not limitation, nuclear spectroscopy logs, spectral gamma ray logs, nuclear magnetic resonance logs, dielectric logs, sonic logs, image logs, and the like.

At step 22, the method performs HD well interpretation. This may include core analyses such as routine and special core analysis. It will include determining the geographical and geological distribution of HD wells. This involves analyzing the geographical distribution of HD wells to understand coverage of HD wells in a developmental area and lateral heterogeneity in geology. It also analyzes the vertical coverage of the logging and coring interval of HD wells to understand the data coverage of different geological formations and vertical heterogeneity. In some embodiments, it may include planning for acquiring new well logs and core analysis in new high-definition wells. Any spatial and vertical gaps identified previously can be used to identify opportunities for acquiring new data in new high-definition wells. Building robust high-definition petrophysical models in individual high-definition wells is an iterative process. It may be performed as follows:

1. Determining minerals and organic matter
i. Choose mineral group to be included in petrophysical model based on existing core analysis and geological knowledge. Typical examples include Illite-Smectite-Chlorite . . . (Clay), Quartz-Feldspar-Mica (QFM), Calcite-Dolomite-Ankerite (Carbonate), Kerogen-Bitumen (Organic Matter), Pyrite (Sulfides).
ii. Choose minerals to be solved in petrophysical model based on quantitative mineralogy measurement from available core data. For instance, we can choose to solve for Quartz and Plagioclase within QFM, Illite representing clays, calcite and Fe-rich dolomite in carbonates, kerogen and pyrites.
iii. Establish potential mineral correlation or constraint from core measurement
   a. Quartz vs plagioclase
   b. Calcite vs Fe-rich dolomite
   c. Pyrite vs organic matter
iv. Choose appropriate basic logs and elemental logs from nuclear spectroscopy logs to drive the mineral solution
   a. Al, Fe, K, Thor for clay
   b. Ca, Mg, Fe for Calcite and Fe-rich dolomite
   c. Fe, S for Pyrite
   d. Ca, S for anhydrite
   e. Si, K, Na, Ca for Quartz, K-feldspar, Na-Feldspar, Ca-Feldspar
   f. C, Uranium for organic matter
v. Calibrate basic and elemental log response of each mineral
   a. based on advanced core analysis that combines core chemistry and mineralogy such as BestRock whenever available.
   b. based on existing database provided by logging vendors

2. Selecting fluid(s) to be included in the petrophysical model at various distances from well bore and determine their petrophysical properties
i. Depending on formation and mud properties, decide whether to solve for both flushed and unflushed zone, or just the unflushed zone.
ii. Gather information about mud type, weight, composition for each well and logging interval
iii. Determine water and hydrocarbon properties such as salinity, AIP, GOR from acquired fluid samples, produced fluids, PVT analysis, etc. Choose fluid types including water, oil, gas, or condensate
iv. Calculate petrophysical properties of formation water based on temperature, pressure, water salinity
V. Calculate petrophysical properties of hydrocarbon properties based on temperature, pressure, oil gravity, gas-oil ratio (GOR), etc.

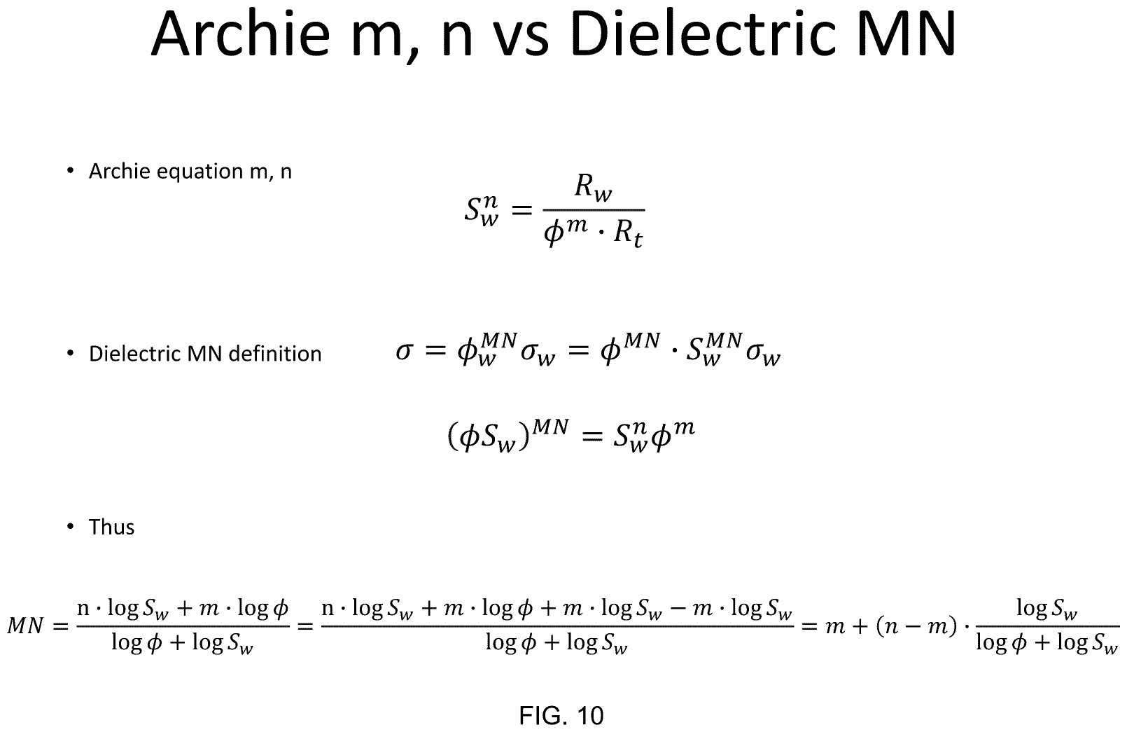

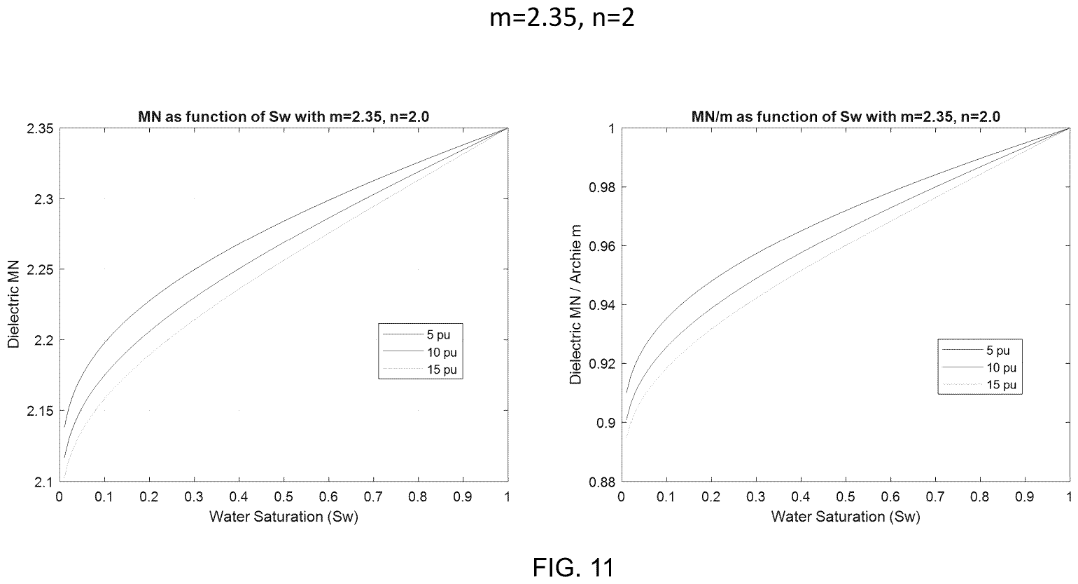

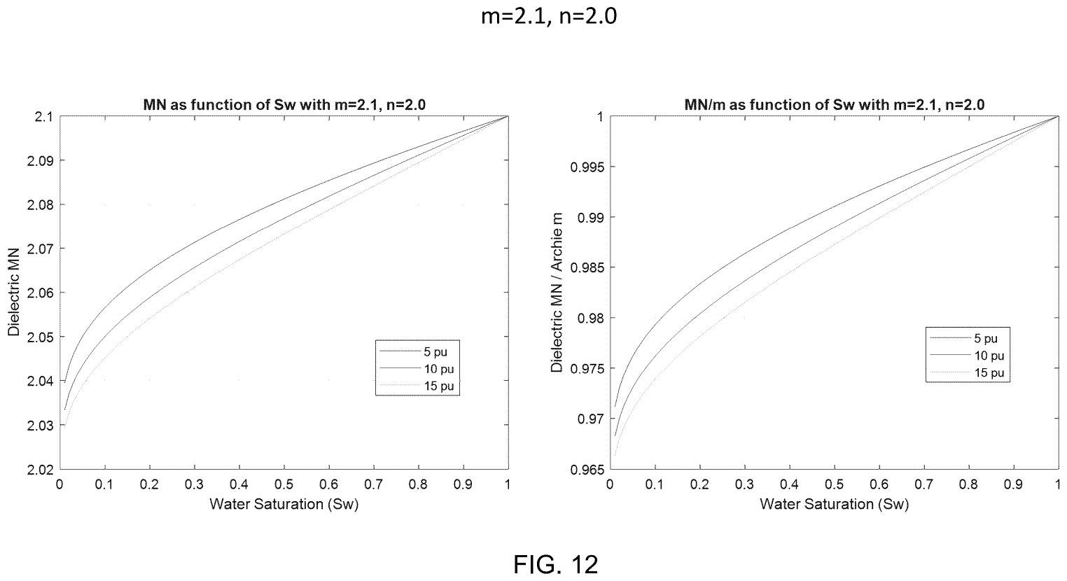

3. Choosing appropriate resistivity-based saturation equations and parameters
i. Choose one or multiple resistivity-based equations to solve for water saturation based on reservoir or formations
ii. Calibrate cation exchange capacity (CEC) from core measurement if available
iii. Calibrate archie parameters such as m, n from core measurement if available.
iv. Archie parameter “m” can also be constrained by water-phase tortuosity parameter “MN” from dielectric dispersion logging. (See FIG. 10. FIG. 11, and FIG. 12 for details)

4. Calculating an initial petrophysical interpretation by minimizing the predicted and measured log response. Such multimineral analysis provide volumetric and gravimetric composition of subsurface formations and reconstructed log response.

5. Iteratively calibrate petrophysical parameters by comparing petrophysical interpretation with other advanced logs and core measurement whenever available
i. Compare and calibrate total porosity from HD results with NMR total porosity, or routine core analysis for porosity.
ii. Compare and calibrate grain density from HD results with grain density from nuclear spectroscopy logs and from core measurement
iii. Compare and calibrate clay-bound water from HD results with NMR or core clay-bound water. This is used to calibrate CEC values used in resistivity-based saturation equation in HD models.
iv. Compare and calibrate water-filled porosity and water saturation from HD results with Dielectric logs or saturation from core measurement

6. Above petrophysical model can be built as one model for entire logging interval or one for each geological formation

7. Iteration steps above to reach finalized petrophysical model and interpretation for individual HD well

In an embodiment, HD models established in one HD well are tested in nearby wells to verify its applicability. HD wells that can be interpreted by the same HD model can be grouped together and define areas of HD model applicability. If necessary, new HD models can be built using above steps. HD models established in one geological area may be tested in HD wells located in anther geological areas to verify its applicability. HD interpretation results can be used for landing point optimization, rock mechanical properties, regional analysis, problem solving, and other detailed interpretations.

At step 24, the process 200 builds a regional model based on the HD well interpretation. These robust regional petrophysical models are based on calibration from the HD wells. The HD well interpretation results serve as continuous calibration throughout the logging interval, extending the calibration at the core points. This step fully utilizes the overdetermined log inputs and core (such as BestRock if available) from HD models to calibrate mineral response for basic logs. This allows the process to establish mineral correlations, constraints, and trends based on HD model and apply to regional models. This step may be another iterative step, iteratively verifying regional models and determining applicable boundaries.

At step 26, the process 200 applies the regional petrophysical model to wells with basic logging suites. The regional petrophysical model takes basic logging suites, such as gamma ray, neutron porosity, bulk density, and resistivity, as inputs. With calibrated log response parameters, regional petrophysical model predicts log responses from an initial mineral and fluid volumes. By varying the volume of each rock and fluid constituents, the difference between predicted and measured log responses is minimized under established mineral and fluids constraints to determine the most likely volumetric composition of the subsurface formation. Some of the basic logging suites, such as gamma ray, neutron density, and photoelectric factor, may require detailed environmental corrections, and/or normalization by advanced algorithms such as that disclosed in U.S. patent application Ser. No. 17/963,559, Multivariate Normalization of Well Logs Using Probability Distribution Modeling, before being used in the regional petrophysical model. The regional petrophysical model results will eventually be delivered to earth scientists, reservoir, production, completion engineers.

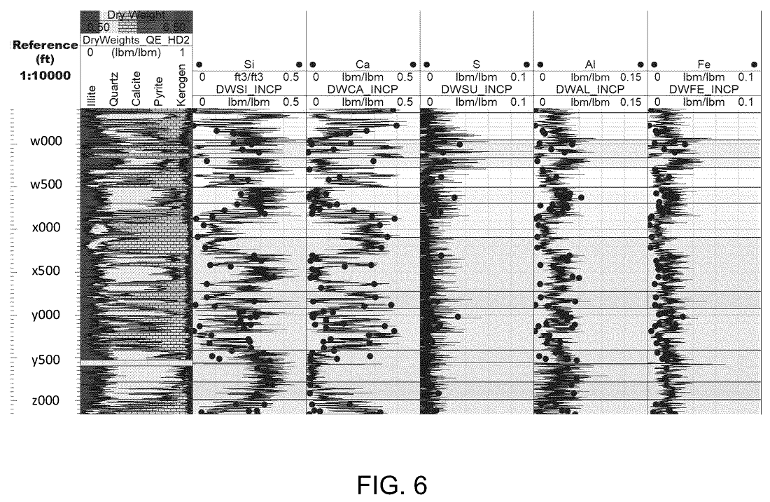

FIG. 6 is a comparison of elemental dry weights from nuclear spectroscopy logs (continuous curves) with those measured by core chemistry (solid dots). Whenever available, direct comparison between elemental dry weights and core chemistry is a direct quality control for nuclear spectroscopy logs. From this comparison, elemental dry-weight logs with sufficient data quality can be included as inputs for HD petrophysical interpretation.

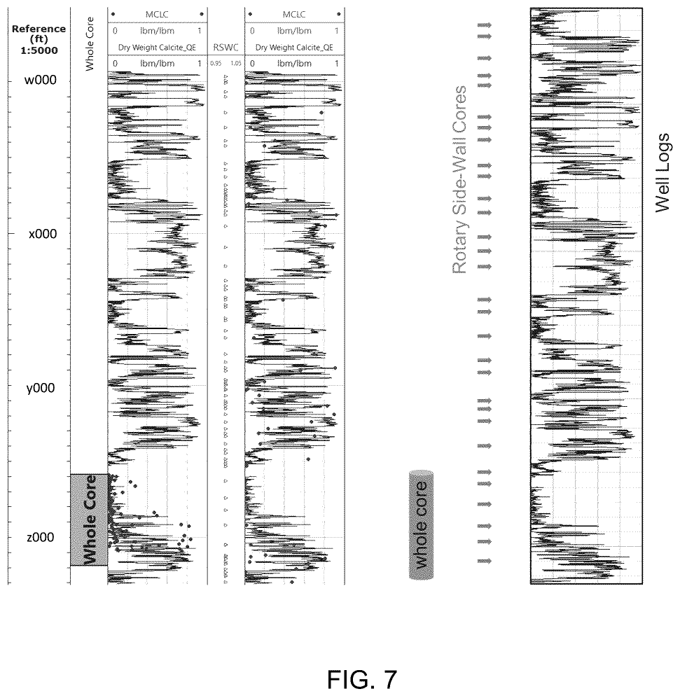

FIG. 7 is a comparison of calibration coverage among whole core, rotary side-wall cores, and well logs. The left panel shows the weight fraction of calcite in comparison with the same quantity measured in core plugs taken from whole core and from rotatory side-wall cores (RSWC). The right panel illustrates the depth coverage of whole core, RSWC, and well logs.

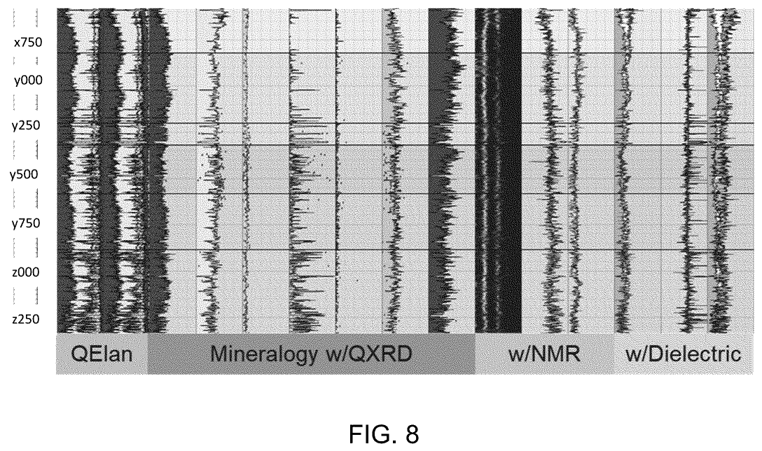

FIG. 8 is an example of high-definition well interpretation results integrating nuclear spectroscopy, NMR, and dielectric logs, and calibration with core mineralogy, porosity, and saturation measurements.

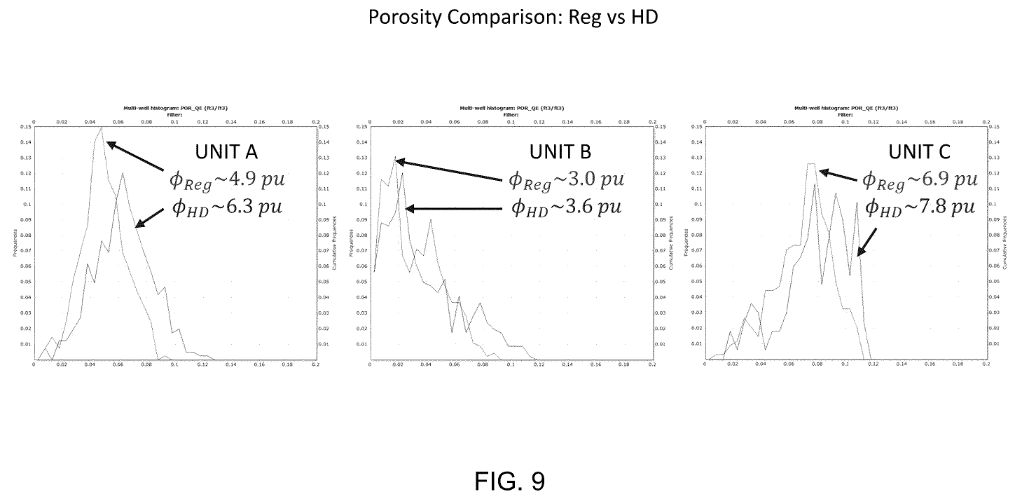

FIG. 9 is an example of porosity comparison between high-definition and current regional models shown as frequency plots of total porosity.

FIG. 10 is derivation of the relationship between water-phase tortuosity parameter “MN” from dielectric dispersion logging and Archie's parameter cementation exponent (m) and saturation exponent (n).

FIG. 11 is numerical calculation of MN as a function of water saturation with typical porosity values (5, 10, 15 porosity units) under specific assumption of m=2.35, n=2.

FIG. 12 is numerical calculation of MN as a function of water saturation with typical porosity values (5, 10, 15 porosity units) under specific assumption of m=2.1, n=2.

While particular embodiments are described above, it will be understood it is not intended to limit the invention to these particular embodiments. On the contrary, the invention includes alternatives, modifications and equivalents that are within the spirit and scope of the appended claims. Numerous specific details are set forth in order to provide a thorough understanding of the subject matter presented herein. But it will be apparent to one of ordinary skill in the art that the subject matter may be practiced without these specific details. In other instances, well-known methods, procedures, components, and circuits have not been described in detail so as not to unnecessarily obscure aspects of the embodiments.

The terminology used in the description of the invention herein is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, operations, elements, and/or components, but do not preclude the presence or addition of one or more other features, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

Although some of the various drawings illustrate a number of logical stages in a particular order, stages that are not order dependent may be reordered and other stages may be combined or broken out. While some reordering or other groupings are specifically mentioned, others will be obvious to those of ordinary skill in the art and so do not present an exhaustive list of alternatives. Moreover, it should be recognized that the stages could be implemented in hardware, firmware, software or any combination thereof.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as are suited to the particular use contemplated.

What is claimed is:

1. A computer-implemented method of generating a regional petrophysical model, comprising:
a. receiving, at one or more processors, high-definition (HD) well logs;
b. building at least one HD petrophysical model based on the HD well logs; and
c. building the regional petrophysical model based on the at least one HD petrophysical model.

2. The method of claim 1 further comprising applying the regional petrophysical model to wells with basic logging suites.

3. The method of claim 1 wherein the building at least one HD petrophysical model comprises:
a. selecting minerals and organic matter to be included in the at least one HD petrophysical model;
b. selecting one or more fluids to be included in the at least one HD petrophysical model and determining petrophysical properties of the one or more fluids;
c. choosing resistivity-based saturation equations and parameters;
d. generating predicted log responses by inputting the minerals and organic matter and the petrophysical properties of the one or more fluids into the resistivity-based saturation equations and parameters;
e. calculating a HD petrophysical interpretation by minimizing differences between the predicted log responses and the HD well logs;
f. calibrating petrophysical parameters by comparing the HD petrophysical interpretation with other advanced logs and core measurement; and
g. repeating steps a-f to generate the at least one HD petrophysical model that best matches the HD well logs.

4. The method of claim 3 wherein the minerals and organic matter are selected based on core analysis and geological knowledge.

5. The method of claim 3 wherein the one or more fluids are selected from water, oil, gas, and condensate.

6. The method of claim 1 wherein the at least one HD petrophysical model is for an entire logging interval or for a geological formation.

7. A computer system, comprising:
one or more processors;
memory; and
one or more programs, wherein the one or more programs are stored in the memory and configured to be executed by the one or more processors, the one or more programs including instructions that when executed by the one or more processors cause the system to:
a. receive, at the one or more processors, high-definition (HD) well logs;
b. build at least one HD petrophysical model based on the HD well logs; and
c. build a regional petrophysical model based on the at least one HD petrophysical model.

8. The system of claim 7 further comprising instructions that when executed by the one or more processors cause the system to apply the regional petrophysical model to wells with basic logging suites.

9. The system of claim 7 wherein the building at least one HD petrophysical model comprises:
a. selecting minerals and organic matter to be included in the at least one HD petrophysical model;
b. selecting one or more fluids to be included in the at least one HD petrophysical model and determining petrophysical properties of the one or more fluids;
c. choosing resistivity-based saturation equations and parameters;
d. generating predicted log responses by inputting the minerals and organic matter and the petrophysical properties of the one or more fluids into the resistivity-based saturation equations and parameters;
e. calculating a HD petrophysical interpretation by minimizing differences between the predicted log responses and the HD well logs;
f. calibrating petrophysical parameters by comparing the HD petrophysical interpretation with other advanced logs and core measurement; and
g. repeating steps a-f to generate the at least one HD petrophysical model that best matches the HD well logs.

10. The system of claim 9 wherein the minerals and organic matter are selected based on core analysis and geological knowledge.

11. The system of claim 9 wherein the one or more fluids are selected from water, oil, gas, and condensate.

12. The system of claim 7 wherein the at least one HD petrophysical model is for an entire logging interval or for a geological formation.
